(19) United States
(12) Patent Application Publication
(10) Pub. No.: US 2025/0241835 A1
(43) Pub. Date: Jul. 31, 2025

(54) Hair Treatment Compositions With Amine Derivatives

(71) Applicant: ELC Management LLC, Melville, NY (US)

(72) Inventors: Jeanna Zguris, North Oaks, MN (US); Daniel Nowlan, III, Hugo, MN (US); Xiuhong Zhai, Vadnais Heights, MN (US)

(21) Appl. No.: 19/042,195

(22) Filed: Jan. 31, 2025

Related U.S. Application Data
(62) Division of application No. 17/656,998, filed on Mar. 29, 2022.

(51) Int. Cl. A61K 8/41; A61K 8/22; A61Q 5/10

(52) U.S. Cl. CPC A61K 8/416; A61K 8/22; A61Q 5/10; A61K 2800/4324; A61K 2800/5922; A61K 2800/88

(57) ABSTRACT

Disclosed is the use of certain alkanolamines, such as 2-dimethyl-amino-2-methyl-1-propanol, for use in hair treatment compositions as a replacement for some or all ammonium hydroxide. When used as hair colorant alkalizers, these alkanolamines demonstrate reduced hair fiber damage, effective color lifting, and significant improvement in cytotoxicity and malodor compared to well known replacements for ammonium hydroxide.

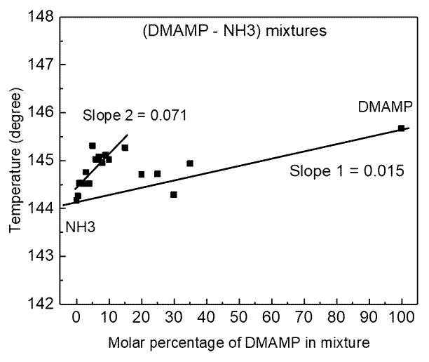

### FIELD OF THE INVENTION

The present invention is in the field of hair treatment applications. Specifically, it is concerned with alternatives to ammonium hydroxide for softening and swelling the cuticle of the hair, and for enabling penetration of reagents and hair-benefit actives into the cortex.

### BACKGROUND

### Hair Structure

Human hair fiber is generally understood as having an outermost layer, called the cuticle. The cuticle comprises about 6-12 layers of overlapping, flattened keratinocytes that are arranged in a “fish scale” arrangement in the longitudinal direction of the hair fiber. The overlapping cellular arrangement permits the cells to slide past each other, which gives hair fibers a high degree of flexibility without breaking. The cuticle layers also regulate the amount of water within the hair shaft. The outermost surface of the cuticle is coated with a lipid substance that renders the surface of the hair hydrophobic. Also, the fish scale arrangement of the cuticle and the lipid coating confer barrier properties to hair fiber. A second layer of hair fiber, below the cuticle, is the cortex. Natural dye, called melanin, is found here. Due to the semi-transparent nature of the cuticle, the melanin in the cortex is normally visible. The cells of the cortex form a matrix that supports keratin protein structures. In the cortex, protein filaments made of long keratin chains are the main structural component of hair. These keratin chains are rich in the sulfur-containing amino acid, cysteine, which forms permanent, thermally stable crosslinking in the form of disulfide bridges between keratin chains. Human hair is approximately 14-20% cysteine. The extensive disulfide bonding of cysteine gives hair approximately one-third of its strength, and makes hair generally insoluble, except in specific dissociating or reducing agents.

### Softening the Cuticle

The present invention is concerned with softening and swelling the cuticle of the hair, for any purpose, such as, but not limited to relaxing, straightening, perming, strengthening and coloring the hair. In various types of hair treatment where swelling and loosening of the cuticle is required, ammonia (in solution) is considered the ‘gold standard’. Ammonium hydroxide, an alkalizing agent, raises the pH of hair, causing the hair cuticles to swell and loosen so that actives and/or reagents can penetrate into the hair. However, the use of ammonia has a number of drawbacks. For example, when in use, ammonia gas readily escapes into the ambient environment, giving off a strong malodor, as well as irritating the skin, eyes, nose and throat. These adverse effects may be experienced by the person whose hair is being treated, as well as by the person providing the treatment. Also, ammonia is known to cause damage to the hair through breaking of peptide bonds. For this reason, research into alternative cuticle penetration methods has been ongoing for several decades, with mixed results. For example, because of their low odor, aminomethyl propanol (AMP) and monoethanolamine (MEA) have been used as replacements for ammonium hydroxide. Both molecules are known to be used in cosmetic formulations as a pH buffer. In terms of their action on hair, the amine functional group, NH₂, reacts similarly to ammonia (NH₃) in an ammonium hydroxide solution, while significantly reducing the ammonia odor. Nevertheless, a substantial increase in hair fiber damage has been associated with AMP and MEA, and this remains a major concern in the field. In fact, until now, no treatment has been found that is as effective at opening up the cuticle as ammonium hydroxide, while also avoiding or significantly reducing the adverse effects of malodor and excessive hair damage.

### Hair Coloring Treatments

While the principles of the invention may pertain to various types of hair treatments, the invention is described herein, in terms of hair coloring treatments.

Coloring of human hair is a very popular cosmetic treatment. Presently, there are four basic types of hair color treatments, classified according to color retention. Temporary and semi-permanent are non-oxidative treatments that employ colored dyes that are deposited on the surface of the hair cuticle. Temporary hair dyeing is used to color their hair for a short time, such as one day. This type of hair color may be achieved with basic dyes, acid dyes, disperse dyes, pigments or metallized dyes. Unable to penetrate the hair due to their molecular size, and with little affinity for the hair, temporary dyes typically wash out with a single wash. In contrast, semi-permanent dye molecules are smaller, and may display some affinity for the hair. The smaller size allows the dye to penetrate into the cuticle, and it is even possible that some of the dye will reach the cortex. Nevertheless, an alkalizer is sometimes used in semi-permanent treatments to facilitate penetration through the cuticle. For this reason, the present invention may find use in semi-permanent hair coloring. As a result of penetrating the cuticle, semi-permanent dyes require about six to twelve shampoos to rinse out. Temporary and semi-permanent hair coloring products are available as lotions, gels, shampoos, liquid solutions, emulsions and mousses.

Permanent hair color treatments provide color that does not wash out with shampooing, and lasts effectively until the treated hair is grown out. The “dyes” in commercial coloring products are actually colorless dye precursors that are small enough to migrate under the swollen cuticle, and diffuse into the cortex. Inside the hair cortex, the precursors undergo a series of redox reactions to develop the final color. In the field of oxidative hair dyeing, we generally speak of two classes of dye precursor molecules: oxidation bases (also known as primary intermediates) and reaction modifiers (also known as couplers or secondary intermediates). By design, the redox potential of the primary intermediate is more favorable for oxidation than the secondary intermediate, such that the primary intermediate will be oxidized first. The weaker oxidation potential means that secondary intermediates alone are capable of producing only slight coloring, but may be used to contribute highlights. Primary intermediates oxidize to highly reactive species that proceed to react with the electron-rich secondary intermediates to form a colorless transient intermediate, called a leuco dye. The leuco dye is rapidly oxidized to a final colored conjugated dye. Due to their size, the conjugated dye molecules resist being rinsed out of the cortex.

In general, the primary and secondary intermediates are of three aromatic types: aromatic diamines, aminophenols, and phenols. The primary intermediates are aromatic diamines and aminophenols where the substituted amino or hydroxy group is located in the para or ortho position, with respect to the amino group. This positioning confers the property of easy oxidation. Primary intermediates are capable of forming quinone, semi-quinone, and imin-quinone structures. Examples of compounds that have found use as primary intermediates include: p-phenylenediamine (PPD), 2-methyl-p-phenylenediamine (PTD), p-aminophenol (PAP), 1,4-dihydroxybenzene, N,N-bis-(2-hydroxyethyl)-p-phenylenediamine, 4,5-diamino-1-(2-hydroxyethyl) pyrazole, 2,4,5,6-tetraaminopyrimidine, o-aminophenol, catechol, and 1,2-benzediamine, and others. Common modifiers are aromatic m-diamines, m-aminophenols, and m-polyphenols. With substituents in a meta position, these molecules are less easily oxidized. Examples include: m-phenylenediamine, 2,4 resorcinol-diaminoanisole, m-chlororesorcinol, m-aminophenol, resorcinol, 2-methyl resorcinol, 1-naphthol, 4-amino-2-hydroxytoluene, and 1,3-benzenediamine.

Two other essential components of an oxidative hair dye system are the alkalizing agent and the oxidizing agent. Both perform multiple functions. For example, as noted above, dye precursors must be able to penetrate into the hair cortex. To facilitate that process, an alkalizing agent (usually ammonium hydroxide) is used to soften and swell the cuticle. In addition, the alkalizer also raises the pH of the cortex environment (to about pH 9-11) which enhances the reactivity of the oxidizing agent. The oxidizing agent (also known as a developer, usually hydrogen peroxide, H₂O₂) oxidizes the primary intermediate to initiate a cascade of oxidation reactions that transform colorless precursor dyes into the final colored complex. At the same time, however, the alkalizing agent converts some of the H₂O₂ to OOH⁻. OOH⁻ is a very reactive depigmenting reagent that neutralizes natural hair melanin or any previously applied oxidative hair color, so that the newly applied color can show through without distortion.

Demi-permanent hair coloring is another treatment where the present invention will find application. Demi-permanent hair color, which lasts for about 20-24 shampoos, occupies an intermediate position between semi-permanent and permanent hair color. Demi-permanent hair color treatments utilize a mix of semi-permanent dyes and dye precursors typical of permanent color treatments. The dyes are mixed with an alkalizing agent (such as monoethanolamine MEA or aminomethylpropanol AMP) that swells the cuticle less efficiently than ammonia. Colorless dye precursors penetrate the outer cuticle, and some is able to enter the cortex, where the precursor molecules then combine to create larger color molecules that resist being washed out. As in permanent dyeing, hydrogen peroxide is used, but at lower concentrations. As a result, the pre-existing hair color is not appreciably lifted. Therefore, this type of dye works well for adding darker colors to hair.

### SUMMARY

The present invention is concerned with compositions and methods for softening and swelling the cuticle of the hair. The compositions comprise certain amine derivatives that feature electron donors/acceptors, making them useful as keratin compatible alkalizing agents for softening and swelling the cuticle of the hair.

### DESCRIPTION OF THE FIGURE

The lone FIGURE displays data of the denaturation temperature of an alkalizer composition comprising a combination of 2-Dimethyl-amino-2-methyl-1-propanol (D M AMP) and NH₃.

### DETAILED DESCRIPTION

Except where otherwise explicitly indicated, all concentrations of materials and conditions of reaction, are to be understood as modified by the word “about.”

All concentrations are presented as percentages by weight of the final composition, unless otherwise specified.

The term “Comprising” and the like, mean that a list of elements may not be limited to those explicitly recited.

Specific examples set forth herein are illustrative only, and the present invention is not limited to those mentioned examples.

### Alkalizing Agents

It can be shown that certain C3-C6 alkanolamines that feature electron donors/acceptors (as the case may be) are useful as alkalizing agents in oxidative and non-oxidative hair coloring applications, either alone or in combination. Alkanolamines are comprised of an alkane backbone that has amino and a hydroxyl functional groups. These relatively large, organic molecules are not as volatile as ammonia. However, like ammonia, alkanolamines, in general, are able to create a strongly basic environment that is potentially damaging to hair and skin cells. It is generally thought that the amine group is responsible for damage to the hair. In fact, depending on the concentration required to reproduce the benefits of ammonia in hair treatment applications, some alkanolamines may produce more or less odor and damage than ammonia. Eleven alkalizing agents that are of particular interest, here, are shown in Table 1 (not including ammonium hydroxide, MEA and AMP which are included for comparison only).

TABLE 1
Alkalizing Agents

| | | | Molecular | |
|---|---|---|---|---|
| | | Structural formula | Weight for | |
| Alkalizer | Molecular | for active | active | |
| (INCI name) | formula | component | component | pKa |
| Ammonium hydroxide | NH₄OH | 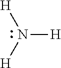 | 17.03 | 9.24 |
| Monoethanol- amine (MEA) | C₂H₇NO | 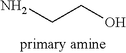 | 61.08 | 9.50 |
| Aminomethyl propanol (AMP) | C₄H₁₁NO | 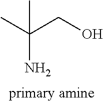 | 89.13 | 9.82 |
| 2-Dimethyl- amino-2-methyl- 1-propanol (DMAMP) | C₆H₁₅NO | 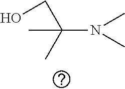 | 117.19 | 10.2 |
| 2-Amino-2- methyl-1,3- propanediol (AMPD) | C₄H₁₁NO₂ | 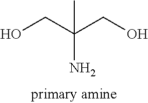 | 105.14 | 8.80 |
| 2-Amino-2- ethyl-1,3- propanediol (AEPD) | C₅H₁₃NO₂ | 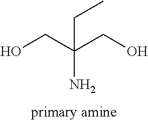 | 119.14 | 8.80 |
| 3-Amino-1- propanol (AP) | C₃H₉NO | 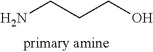 | 75.11 | 9.96 |
| 3-Dimethyl- amino-1- propanol (DMAP) | C₅H₁₃NO | 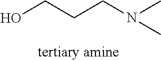 | 103.16 | 9.27 |
| 3-(Dimethyl- amino)-1,2- propandiol (DMAPD) | C₅H₁₃NO₂ | 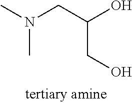 | 119.16 | — |
| 3-Amino-1,2- propanediol (Isoserinol) | C₃H₉NO₂ | 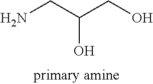 | 91.11 | — |
| 2-Amino-1,3- propanediol (Serinol) | C₃H₉NO₂ | 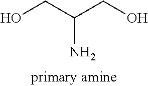 | 91.11 | — |
| Diethanolamine (DEA) | C₄H₁₀NO₂ | 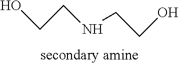 | 105.14 | 8.88 |
| Triethanolamine (TEA) | C₆H₁₅NO₃ | 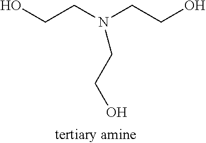 | 149.19 | 7.8 |
| Tromethamine (a.k.a. Tris) | C₄H₁₁NO₃ | 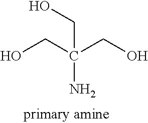 | 121.14 | 8.1 |

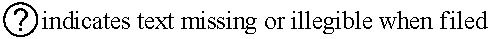

In determining which of these eleven compounds or combinations thereof may offer performance benefits over ammonia, aminomethyl propanol (AMP) and monoethanolamine (MEA), a study was made of the ability of each of the eleven compounds to lift natural color out of the hair, the degree of damage caused by applying the compounds to the hair, and the degree of malodor. These results will be discussed below.

In alkalizing compositions of the invention, the total amount of all alkanolamine alkalizing agents will typically range from about 0.001 to 25%; for example from about 0.4% to about 20%; for example from about 1% to about 15%; for example from about 2% to about 12.5%; for example from about 3% to about 10%. If ammonium hydroxide is used in combination with an alkanolamine identified herein, then the concentration of ammonium hydroxide should be limited to about 0.01% to 14%.

### Oxidative Hair Dye Products

In practice, an oxidative hair-dye product consists of two containers, a first containing (I) an alkalizer composition, and a second containing (II) an oxidizing agent composition. These are mixed shortly before application to the hair. The mixture may be referred to as the on-hair product.

### I. The Alkalizer Composition

Alkalizer compositions of the invention comprise an aqueous solution of one or more alkalizing agents shown in Table 1, and one or more oxidative dyes. Optionally, various auxiliary ingredients may be included which impart a benefit to the alkalizer composition or to the hair.

### Oxidative Dyes

Alkalizer compositions according to the present invention comprise one or more primary intermediates that are operable, when combined with an oxidizing agent, to impart color to the hair. Optionally, the alkalizer compositions may also comprise one or more couplers.

### Primary Intermediates

Primary intermediates may generally be present in the alkalizer composition in amounts ranging from about 0.001 to 25%, preferably from about 0.005 to 20%, more preferably from about 0.01 to 15% by weight of the total alkalizer composition. Such primary intermediates include ortho or para substituted aminophenols or phenylenediamines, such as para-phenylenediamines of the formula:

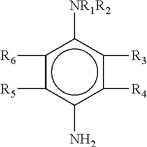

wherein R1 and R2 are each independently hydrogen, C1-6 alkyl, or C1-6 alkyl substituted with hydroxy, methoxy, methylsulphonylamino, furfuryl, aminocarbonyl, unsubstituted phenyl, or amino substituted phenyl groups; and R3, R4, R5, and R6 are each independently hydrogen, C1-6 alkyl, C1-6 alkoxy, halogen, or C1-6 alkyl substituted with one or more amino or hydroxyl groups. Such primary intermediates include para-phenylenediamine (PPD), 2-methyl-1,4-diaminobenzene, 2,6-dimethyl-1,4-diaminobenzene, 2,5-dimethyl-1,4-diaminobenzene, 2,3-dimethyl-1,4-diaminobenzene, 2-chloro-1,4-diaminobenzene, 2-methoxy-1,4-diaminobenzene, 1-phenylamino-4-aminobenzene, 1-dimethylamino-4-aminobenzene, 1-diethylamino-4-aminobenzene, 2-isopropyl-1,4-diaminobenzene, 1-hydroxypropylamino-4-aminobenzene, 2,6-dimethyl-3-methoxy-1,4-diaminobenzene, 1-amino-4-hydroxybenzene, 1-bis(beta-hydroxyethyl)amino-4-aminobenzene, 1-methoxyethylamino-4-aminobenzene, 2-hydroxymethyl-1,4-diaminobenzene, 2-hydroxyethyl-1,4-diaminobenzene, and derivatives thereof, and acid or basic salts thereof. Also suitable are various types of pyrimidines such as 2,3,4,5-tetraaminopyrimidine sulfate and 2,5,6-triamino-4-pyrimidinol-sulfate. Preferred primary intermediates are p-phenylenediamine, p-aminophenol, o-aminophenol, N,N-bis(2-hydroxyethyl)-p-phenylenediamine, 2,5-diaminotoluene, their salts and mixtures thereof.

### Couplers

If present, the color couplers may range from about 0.0001-10%, more preferably about 0.0005-8%, most preferably about 0.001-7% by weight of the total alkalizer composition. Such color couplers include, for example, those having the general formula:

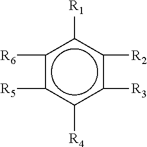

wherein R1 is unsubstituted hydroxy or amino, or hydroxy or amino substituted with one or more C1-6 hydroxyalkyl groups; R3 and R5 are each independently hydrogen, hydroxy, amino, or amino substituted with C1-6 alkyl, C1-6 alkoxy, or C1-6 hydroxyalkyl group; and R2, R4, and R6 are each independently hydrogen, C1-6 alkoxy, C1-6 hydroxyalkyl, or C1-6 alkyl. Alternatively, R3 and R4 together may form a methylenedioxy or ethylenedioxy group. Examples of such compounds include meta-derivatives such as phenols, catechol, meta-aminophenols, meta-phenylenediamines, and the like, which may be unsubstituted, or substituted on the amino group or benzene ring with alkyl, hydroxyalkyl, alkylamino groups, and the like. Suitable couplers include m-aminophenol, 2,4-diaminotoluene, 4-amino, 2-hydroxytoluene, phenyl methyl pyrazolone, 1,3-diaminobenzene, 6-methoxy-1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene. 6-ethoxy-1-bis(beta-hydroxyethyl)amino-3-aminobenzene, 6-hydroxyethyl-1,3-diaminobenzene, 3,4-methylenedioxyphenol, 3,4-methylenedioxy-1-[(beta-hydroxyethyl)amino]benzene, 1-methoxy-2-amino-4-[(beta-hydroxyethyl)amino]benzene, 1-hydroxy-3-(dimethylamino)benzene, 6-methyl-1-hydroxy-3[(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, 2,4-diamioniphenoxyethanol, and mixtures thereof.

### Auxiliary Ingredients

### Reducing Agents and Antioxidants

The alkalizer composition may further comprise one or more reducing agents and/or one or more antioxidants. Reducing agents and antioxidants are able to stabilize the composition by inhibiting reactions between the primary intermediates and couplers as well as the onset of oxidation through exposure to atmospheric oxygen. A commonly used reducing agent is sodium metabisulfite, which may be used in the range of 0.1% to 5%, by weight of the alkalizer composition. Water soluble antioxidants include erythorbic acid. If the alkalizer composition is an emulsion, then an oil-soluble antioxidant, such as t-butylquinone may be useful. Antioxidants may typically comprise 0.1% to 5% by weight of the alkalizer composition.

### Emollient Oils

If desired the alkalizer composition may contain one or more emollient oils. Such oils will provide a conditioning effect to the hair. If present, such oils may range from about 0.001 to 45% preferably from about 0.01 to 40%, more preferably from about 0.1 to 35% by weight of the alkalizer composition. Suitable oils include silicones such as dimethicone, phenyl silicones, fatty alkyl silicones such as cetyl or stearyl dimethicone, or silicone surfactants which are generally referred to as dimethicone copolyols, or cetyl dimethicone copolyol. Also suitable are various animal, vegetable, or mineral oils derived from plants or animals, or synthetic oils. Examples include oils from sunflower, castor seeds, orange, lemon, jojoba, mineral oil, and the like.

### Surfactants

The alkalizer composition may comprise one or more surfactants. Suitable surfactants include well known cosmetically acceptable anionic, nonionic, amphoteric and cationic surfactants, and the like. If present, surfactants may range from about 0.001-50%, preferably about 0.005-45%, more preferably about 0.1-40% by weight of the alkalizer composition.

### Polar Solvents

The alkalizer composition may also comprise a variety of nonaqueous polar solvents other than water, including mono-, di-, or polyhydric alcohols, and similar water soluble ingredients. If present, such polar solvents may range from about 0.01-25%, preferably about 0.05-15%, more preferably about 0.1-10% by weight of the first composition of polar solvent. Examples of suitable monohydric alcohols include ethanol, isopropanol, benzyl alcohol, butanol, pentanol, ethoxyethanol, and the like. Examples of dihydric or polyhydric alcohols, as well as sugars and other types of humectants that may be used, include glycerin, glucose, fructose, mannose, mannitol, maltitol, lactitol, inositol, and the like. Suitable glycols include propylene glycol, butylene glycol, ethylene glycol, polyethylene glycols having from 4 to 250 repeating ethylene glycol units, ethoxydiglycol, and the like.

### Chelating Agents

The alkalizer composition may optionally contain 0.0001-5%, preferably 0.0005-3%, more preferably 0.001-2% of one or more chelating agents which are capable of complexing with and inactivating metallic ions in order to prevent their adverse effects on the stability or effects of the composition. In particular, the chelating agent will chelate the metal ions found in the water and prevent these ions from interfering with the deposition and reaction of the dye with the hair fiber surface. Suitable chelating agents include EDTA and calcium, sodium, or potassium derivatives thereof, HEDTA, sodium citrate, TEA-EDTA, and so on.

### pH Adjusters

It may also be desirable to add small amounts of acids or bases to adjust the pH of the alkalizer composition to the desired pH range, such that the final on-hair product has a pH of from about 8 to about 12. Suitable acids include hydrochloric acid, phosphoric acid, and the like. Suitable bases include sodium hydroxide, ammonium hydroxide, potassium hydroxide, and the like, as well as the basic amino acids (arginine, lysine and histidine). Also suitable are primary, secondary, or tertiary amines and derivatives thereof such as aminomethyl propanol, monoethanolamine, and the like. Suggested ranges of pH adjusters are from about 0.00001-8%, preferably about 0.00005-6%, more preferably about 0.0001-5% by weight of the total alkalizer composition.

### Botanical Ingredients

The alkalizer composition may comprise one or more botanical ingredients. If present, suggested ranges are from about 0.00001-10%, preferably from about 0.0001-8%, more preferably from about 0.0001-5% by weight of the total alkalizer composition. Examples of such ingredients include Camellia Sinensis extract, Camellia Oleifera extract, Vanilla extract, Green Tea extract, Aloe Barbadensis extract, and the like.

### Container for the Alkalizing Composition

The alkalizer composition is preferably stored in a container that is air-tight and made of a material that is oxidation resistant. Preferably such containers are in the form of tubes, jars, bottles, and the like. Preferred, is where the container is a tube, preferably a tube that can be compressed to dispense the alkalizer composition found therein. Suitable tubes may be metallic. Preferred is where the tube is an oxidation resistant aluminum. In the most preferred embodiment, the tube is made from oxidation resistant aluminum having less than 100 ppm of cadmium, mercury, lead, and hexavalent chromium. The closure for the container of the alkalizer composition must prohibit air from oxidizing the contents of the container. A variety of closures are suitable including screw caps, snap off lids, and the like. Preferably the closure is reusable in the event that multiple uses are desired, for example, in a salon environment. Once the container is opened it may be used to dispense the desired amount of alkalizer composition as needed. The container may be re-closed, and stored for hours, days, weeks, or even months, before the remaining contents are used. An alkalizer composition formulated according to the invention and stored in a suitable container can be used, and the remaining contents stored indefinitely. For example, including an antioxidant in the alkalizer composition will enable the container of oxidative hair dye to be used and stored from 1-6 days, or from 1 to 3 weeks, or from 1 to 4 months before it is used again.

### II. The Oxidizing Agent Composition

Immediately prior to application to hair, the alkalizer composition of the invention is combined with an oxidizing agent composition to form a hair-dyeing composition. Aqueous forms of the oxidizing agent composition contain water, generally in an amount ranging from about 65% to 99%, preferably from about 70 to 97%, most preferably from about 70% to 94% by weight of the oxidizing agent composition. Aqueous forms of the oxidizing agent composition may include lotions, creams and gels. Anhydrous forms of the oxidizing agent composition are sometimes used (powders, for example). In addition, the oxidizing agent composition also comprises an oxidizing agent that will react with the precursor dyes present in the alkalizer composition. Most often the oxidizing agent used is hydrogen peroxide, but other peroxides or oxidizing agents may be used such as calcium peroxide, sodium percarbonate and one or more persulfates (i.e. ammonia, potassium and sodium). Preferably the hydrogen peroxide concentration in the oxidizing agent composition ranges from about 1 to 20% by weight of the oxidizing agent composition.

The oxidizing agent composition may typically comprise peroxide stabilizers, such as sodium stannate and pentasodium pentetate. Alternatively, some type of chelating system may be used to maintain the relatively low pH of the oxidizing agent composition. Stabilizers and/or chelating system may comprise 0.01% to 5.0% by weight of the oxidizing agent composition.

### IV. Testing of Alkalizer Compositions

The alkanolamines in Table 1 were tested in a base dye composition (having no dyes, nor dye precursors) to evaluate their suitability as alkalizers. The alkalizer compositions according to the invention, as well as control compositions, were subjected to various analytical techniques, including thermodynamic, optical and tensile analysis, and cytotoxicity testing.

### Hair Sample Preparation

Level 4 mixed-source human hair tresses were purchased from International Hair Importers & Products, Inc (New York). Testing was performed on virgin hair (control), hair that had been treated with ammonium hydroxide, and hair that had been treated with various single alkalizer compounds and combinations thereof, as described herein. Ten grams of freshly made alkalizer composition were mixed with 10 grams of volume 40 (12%) oxidizer developer (Aveda Color Catalyst Conditioning Crème Developer) until a homogenous cream was obtained. The ammonium hydroxide samples were also mixed with volume 40 oxidizer developer.

Approximately 4 grams of cream mixture per gram of hair was applied to sample hair tresses. Each hair tress was then incubated at 37° C. oven for 45 minutes. The hair tresses were rinsed with tap water for 1 minute before applying SDS (sodium dodecyl sulfate) 5% solution. Each hair tress was massaged for 30 seconds in SDS solution. The hair tresses were rinsed again with tap water for 1 minute to wash off all surfactant. The treated tresses were blown dry with a hair dryer on medium/high speed with medium/high heat. Thereafter, the tresses were allowed to air dry at room temperature for 12 hours, before being subjected to differential scanning calorimetry (DSC) and spectrophotometric analysis.

### Alkalizer Compositions Tested

The following base alkalizer composition (without dyes or dye precursors) was used to test each alkalizing agent or combinations thereof.

TABLE 2
Base alkalizer composition

| INCI Name | Percent |
|---|---|
| Cocamide MEA | 10.00 |
| Glyceryl stearate | 4.00 |
| Cetearyl alcohol | 2.50 |
| Steareth-21 | 2.50 |
| Euphorbia cerifera | 2.00 |
| (candelilla) wax | |
| Oleic acid | 1.00 |
| Decyl glucoside | 5.00 |
| Glycerin | 2.00 |
| Erythorbic acid | 0.20 |
| Tetrasodium EDTA | 0.40 |
| Sodium sulfite | 0.20 |
| Water | q.s. |

Table 3 shows the amount of each individual alkalizer that was added to one composition of Table 2 to complete an alkalizer composition. Also shown are the pH, viscosity and alkalinity of the alkalizer composition. The Brookfield LVDVII Pro Viscometer was used to measure the viscosity of the formulation. The measurements were performed at 22° C. with T-F spindle at 6 rpm. All compositions contain the same molar percentage of alkalizer, the water content being adjusted accordingly. Ammonium hydroxide, being the gold standard in alkalizers, serves as a control, and MEA and AMP as common replacements for ammonium hydroxide are included for comparison.

TABLE 3

| | Percent | | | |
|---|---|---|---|---|
| | (by weight of | | | |
| | total alkalizer | | Viscosity | Alkalinity |
| Alkalizer | composition) | pH | (cP × 10⁵) | (ml/g) |
| Ammonium | 6.90 | 10.72 | 5.11 | 2.32 |
| hydroxide (29%)* | | | | |
| MEA (99%)* | 7.17 | 10.85 | 3.02 | 2.35 |
| AMP (95.5%)* | 10.84 | 10.91 | 1.81 | 2.32 |
| DMAMP (78%)* | 17.46 | 11.15 | 0.211 | 2.38 |
| AMPD | 12.30 | 10.28 | 3.84 | 2.49 |
| AEPD (97%)* | 16.58 | 10.33 | 4.47 | 2.40 |
| AP | 8.80 | 11.49 | 3.26 | 3.10 |
| DMAP | 12.08 | 10.58 | 2.77 | 2.32 |
| DMAPD | 14.10 | 10.37 | 3.23 | 2.57 |
| Isoserinol | 10.78 | 10.56 | 3.75 | 2.45 |
| Serinol (97%)* | 11.11 | 10.22 | 10.1 | 2.55 |
| DEA | 12.44 | 10.35 | 5.09 | 2.57 |
| TEA | 17.63 | 9.18 | 2.02 | 2.74 |
| Tris | 14.35 | 9.6 | 4.17 | 2.43 |

*percent active

Various binary combinations of alkalizers were also tested by combining them into the base composition shown in Table 2.

### DSC Analysis

Protein denaturation occurs when proteins lose their secondary, tertiary or quaternary structure by application of some external stress or compound, such as a strong acid or base, a concentrated inorganic salt, an organic solvent (e.g., alcohol or chloroform), or heat, while the peptide bonds between the amino acids (primary structure) are left intact. Denaturation of tertiary structure includes disruption of interactions between amino side chains, such as covalent disulfide bridges between cysteine groups, non-covalent dipole-dipole interactions between polar groups, and Van der Waals interactions between non-polar groups in the side chains. Denaturation of secondary structure means that proteins lose all regular repeating patterns (such as alpha-helix structure and beta-pleated sheets), and adopt a random coil configuration.

It is known that the denaturation of keratin in hair can be detected by differential scanning calorimetry. DSC is a thermal analysis technique used to measure transition temperature and heat of transformation (enthalpy) for endothermic and exothermic reactions. DSC is typically used to measure melting and solidification temperatures at different melting or cooling rates. DSC is sensitive enough to provide information about molecular weight distributions of polymers.

Denaturation measurements were made on virgin hair (control), hair that had been treated with a mixture of ammonium hydroxide and volume 40 oxidizer developer (control), and hair that had been treated with various alkalizer compounds (including volume 40 oxidizer developer), as described above. Also included, for comparison purposes, are hair samples treated with NaOH, which, above a certain concentration, is a very potent alkalizer that induces significant damage in human hair. NaOH is included as a worst damage level indicator.

Measurements were performed using the Mettler Toledo DSC 822e (from Mettler Toledo LLC, Columbus OH), or Discovery DSC 2500 (from TA Instruments. New Castle, DE). The experiments were carried out over a temperature range of 25° C. to 180° C., with a scan rate of 5° C./min under nitrogen protection. DSC samples were prepared by cutting tress samples into pieces (0.1 to 1.0 mm in size) and weighing. The hair samples were mixed with deionized water, and then sealed in high volume pans for at least 6 hours before measuring. The phase transition temperature (keratin denaturation temperature) of each hair sample was analyzed using either STA Re software (Mettler Toledo DSC822e) or TRIOS software (Discovery DSC 2500). Each hair sample was analyzed at least twice, and the average temperature was obtained for data analysis. A higher denaturation temperature indicates that less damage was incurred by the hair as a result of treatment with the mixture of alkalizer composition and oxidizer developer. Results are given in Tables 4A and 4B.

TABLE 4A
DSC; single alkalizer compositions

| | | | % Relative | |
|---|---|---|---|---|
| | | | damage level | |
| | | ΔT | compared | |
| | | (change in | to NaOH | |
| | | denaturation | (T_{Virgin}- | |
| | Denaturation | temperature) | T_{Alkalizer})/ | Damage level |
| | Temperature | T_{Virgin}- | (T_{Virgin}- | normalized |
| Alkalizer | (° C.) | T_{Alkalizer} | T_{NaOH}) | to NH₃•H₂O |
| Virgin Hair | 150.42 | — | — | — |
| NH₃—H₂O | 147.63 | 2.79 | 19.52% | 1 |
| (29%) | | | | |
| NaOH | 136.13 | 14.29 | 100% | 5.12 |
| MEA | 141.96 | 8.46 | 59.20% | 3.03 |
| AMP | 141.83 | 8.59 | 60.11% | 3.08 |
| DMAMP | 147.84 | 2.58 | 18.05% | 0.92 |
| (85%) | | | | |
| AMPD | 145.88 | 4.54 | 31.77% | 1.63 |
| AEPD | 146.25 | 4.17 | 29.18% | 1.49 |
| (97%) | | | | |
| AP | 138.42 | 12 | 83.97% | 4.30 |
| DMAP | 147.80 | 2.62 | 18.33% | 0.94 |
| DMAPD | 149.46 | 0.96 | 6.72% | 0.34 |
| Isoserinol | 144.59 | 5.83 | 40.80% | 2.09 |
| Serinol | 147.23 | 3.19 | 22.32% | 1.14 |
| (97%) | | | | |
| DEA | 148.63 | 1.79 | 12.53% | 0.64 |
| TEA | 150.89 | −0.47 | −3.29% | −0.17 |
| Tris | 149.00 | 1.42 | 9.94% | 0.51 |

The above results may be interpreted in terms structure, intramolecular hydrogen bonding and shielding of the amine group. Table 4B lists the thirteen alkanolamine alkalizers in order from lowest denaturation temperature to highest. In order of importance, the characteristics that may explain the results are the order of the amine (primary, secondary, tertiary); number of OH groups, how many carbon atoms away the OH groups are from the amine, whether or not the nitrogen is flanked by OH and methyl groups.

TABLE 4B
DSC; single alkalizer compositions

| | | | OH group(s): | Flanking |
|---|---|---|---|---|
| | | Number | number of C | of amine |
| | | of OH | atoms away | by OH |
| Alkalizer | Amine order | groups | from amine | groups |
| AP | primary | 1 | 3 | no |
| AMP | primary | 1 | 2 | no |
| MEA | primary | 1 | 2 | no |
| Isoserinol | primary | 2 | 2, 3 | partial |
| AMPD | primary | 2 | 2, 2 | yes |
| AEPD | primary | 2 | 2, 2 | yes |
| (97%) | | | | |
| Serinol | primary | 2 | 2, 2 | yes |
| (97%) | | | | |

Below this line, treated hair samples showed a higher denaturation
temperature than samples treated with ammonium hydroxide

| | | | | |
|---|---|---|---|---|
| DMAP | tertiary | 1 | 3 | no |
| DMAMP | tertiary | 1 | 2 | no |
| (85%) | | | | |
| DEA | secondary | 2 | 2, 2 | yes |
| Tris | primary | 3 | 2, 2, 2 | yes |
| DMAPD | tertiary | 2 | 2, 3 | partial |
| TEA | tertiary | 3 | 2, 2, 2 | yes |

The above results also indicate that AP (3-amino-1-propanol) was the only alkalizer that produced significantly more damage than either of the common replacements for ammonium hydroxide, MEA and AMP. Noticeably, MEA and AMP caused roughly 3 fold higher damage than ammonium hydroxide. Serinol and DMAP performed the closest to ammonium hydroxide, making them suitable replacements for all, most or some of the ammonium hydroxide, at least in terms of denaturation temperature of the hair.

All of the hair samples treated with primary amine alkalizer compositions (except for Tris) show a lower denaturation temperature than the sample treated with an ammonium hydroxide composition, although hair sample treated with Serinol shows only a slightly lower denaturation temperature. However, among the primary amine alkalizers, AMPD, AEPD, Isoserinol and Serinol performed significantly better than MEA and AMP, and may therefore, be considered useful for softening and swelling the cuticle of the hair, and for enabling penetration of reagents and hair-benefit actives into the cortex.

Within the primary amine alkalizers, those with two OH groups performed better than those with only one. Within each of those subgroups, those wherein the OH group or groups were, on average, closer to the amine performed better. Here, the term “closer” means fewer number of intervening carbon atoms. Also of note is whether or not the amine is “flanked” by two or more OH groups within two carbon atoms. The amines in Isoserinol and DMAPD are flanked by two OH groups, but only one of the OH groups is within two carbon atoms.

All of the tertiary and secondary amine alkalizer compositions (TEA, DMAPD, DEA, DMAMP and DMAP) were less damaging to hair than the ammonium hydroxide composition. Tris, a primary amine alkalizer, also performed well. Among all of these alkalizers, those with two or three OH groups performed better than those with only one. Among the secondary and tertiary amine alkalizers, those with more OH groups performed better. Tris is a special case. As a primary amine Tris might have been expected to perform less well. However, having three OH groups all within two carbon atoms of the amine, and partial flanking seems to have contributed to its performance. TEA produced the least damage to the tested hair sample, but a residual coating may form on the hair surface. The following generalization can be made: when the objective is to limit damage to hair, then primary alkanolamines with at least two OH groups, as well as secondary and tertiary alkanolamines, are preferred.

It seems that at least some of the performance of each alkalizer can be attributed to stabilizing or shielding of the amine group as a result of intramolecular hydrogen bonding, especially between the hydrogens of the hydroxyl groups and nitrogen, although some other hydrogen bonding or other effects may also be occurring. In fact, the only difference between AP (the worst performer in DSC testing) and DMAP (a better performer than ammonia) is the two methyl groups on the amine of DMAP, which appear to be stabilizing the amine to a significant degree.

### Color Lifting

Spectrophotometry was used to evaluate changes in color and changes in the appearance of various hair samples, as a result of exposure to various alkalizer compounds and combinations thereof, as described herein. Spectrophotometry can be used to measure the light reflected from a given surface or object. Konica Minolta® CM-600d Spectrophotometer and accompanying SpectraMagic NX software were used to collect data for evaluation of hair tress color. The standard is to express color as three different numerical values (L*, a*, and b*). The values are intended to mimic what is perceived by the human eye. The a* value, which represents the red/green color of the hair sample, and the b* value, which represents the yellow/blue color of the hair sample, are not reported, here. The L* value, however, represents the light/dark intensity of the measurement surface. L* values range from 0 to 100, where 0 is pure black and 100 is pure white. The higher the L* value, the lighter the hair color, and the more effective the alkalizer at lifting natural hair color. Measurements of L* were made on virgin hair (control), hair that had been treated with a mixture of ammonium hydroxide and volume 40 oxidizer developer (control), and hair that had been treated with various alkalizer compounds (including volume 40 oxidizer developer), as described above. Also included, for comparison purposes, are hair samples treated with NaOH, which, above a certain concentration is a very potent alkalizer that is expected to induce a significant loss of melanin. Each hair tress was bound on one end to form a swatch wherein the hair is uniformly distributed along the binding. The measured values of L* are given in Table 5.

TABLE 5
L Values; single alkalizer compositions

| | | | Relative | |
|---|---|---|---|---|
| | | ΔL | lifting level | |
| | | (change in | in percentage | |
| | | color lifting) | (L_{Virgin}- | Lifting level |
| | | L_{Virgin}- | L_{Alkalizer})/ | normalized |
| Alkalizer | L Value | L_{Alkalizer} | (L_{Virgin}-L_{NaOH}) | to NH₃•H₂O |
| Virgin | 19 | — | — | — |
| NH₃—H₂O | 34 | −15 | 85.23% | 1 |
| (29%) | | | | |
| NaOH | 36.6* | −17.6 | 100% | 1.17 |
| AP | 37 | −18 | 102.27% | 1.20 |
| MEA | 36 | −17 | 96.59% | 1.13 |
| Isoserinol | 36 | −17 | 96.59% | 1.13 |
| AMP | 32.5 | −13.5 | 76.70% | 0.90 |
| AMPD | 32 | −13 | 73.86% | 0.87 |
| Serinol | 31 | −12 | 68.18% | 0.80 |
| (97%) | | | | |
| DEA | 30.5 | −11.5 | 65.34% | 0.77 |
| AEPD | 30 | −11 | 62.50% | 0.73 |
| (97%) | | | | |
| DMAP | 27 | −8 | 45.45% | 0.53 |
| Tris | 27 | −8 | 45.45% | 0.53 |
| DMAPD | 25.5 | −6.5 | 36.93% | 0.43 |
| DMAMP | 26 | −7 | 39.77% | 0.47 |
| (85%) | | | | |
| TEA | 23 | −4 | 22.73% | 0.27 |

The results show that the isoserinol/oxidizer mixture and the 3-amino-1-propanol (AP)/oxidizer mixture are better at lifting natural hair color than the ammonium hydroxide/oxidizer mixture (and even similar to or better than NaOH). Furthermore, isoserinol displayed about the same effectiveness as MEA, and significantly better effectiveness than AMP, the two common replacements for ammonium hydroxide. 3-amino-1-propanol (AP) outperformed all of them. The other compositions were less effective at lifting hair color than ammonium hydroxide, MEA and AMP. However, AMPD was almost as good as MEA and ammonium hydroxide. In terms of color lifting, serinol and DMAP, which compared well to ammonium hydroxide vis-à-vis damage, performed significantly less well than ammonium hydroxide. This may suggest a combination of alkalizing agents, such as ammonia, MEA or isoserinol combined with serinol or DMAP, to obtain the benefits of both.

The color lifting results are roughly reversed from the DSC results above in that all of the primary amine alkalizer compositions performed better than the tertiary amine alkalizer compositions, except that Tris was as good as tertiary alkalizer DMAP. Also the primary amine alkalizer, AEPD, was not quite as good as secondary amine alkalizer, DEA, but close.

### Testing Combinations of Alkalizers

We surmised that combinations of alkalizers are likely to combine the benefits of each while mitigating the drawbacks. Based on the above results, select binary combinations of alkalizers were also tested by combining two alkalizer compositions made according to Tables 2 and 3. The first set of combinations all involve NH₃, as follows: AMPD-NH₃, AEPD-NH₃, DMAMP-NH₃, Tris-NH₃ and Serinol-NH₃. These combinations were tested in different mole ratios as shown in the tables 6-10, below.

TABLE 6
AMPD-NH₃

| | Denaturation | |
|---|---|---|
| Mole ratio | Temperature | |
| AMPD/NH₃ | (° C.) | L* value |
| 100% AMPD | 143.45 | 32.1 |
| 90/10 | 143.41 | 31.6 |
| 67/33 | 143.77 | 32.7 |
| 50/50 | 143.80 | 31.5 |
| 33/67 | 144.30 | 32.4 |
| 10/90 | 144.34 | 32.1 |
| 100% NH₃ | 144.10 | 33.6 |
| Virgin hair | 146.76 | 20.0 |

TABLE 7
AEPD-NH₃

| | Denaturation | |
|---|---|---|
| Mole ratio | Temperature | |
| AEPD/NH₃ | (° C.) | L* value |
| 100% AEPD | 146.25 | 30 |
| 75/25 | 146.54 | 32.9 |
| 50/50 | 147.39 | 33.3 |
| 25/75 | 146.96 | 32 |
| 100% NH₃ | 146.92 | 33 |
| Virgin hair | 149.84 | 20.2 |

TABLE 8A
DMAMP-NH₃

| | Denaturation | |
|---|---|---|
| Mole ratio | Temperature | |
| DMAMP/NH₃ | (° C.) | L* value |
| 100% DMAMP | 147.84 | 26 |
| 75/25 | 144.89 | 27 |
| 70/30 | 147.92 | 29 |
| 60/40 | 147.63 | 29 |
| 50/50 | 147.11 | 32 |
| 40/60 | 146.84 | 32.5 |
| 25/75 | 148.55 | 33.5 |
| 10/90 | 148.04 | 33.9 |
| 100% NH₃ | 145.83 | 34 |
| Virgin hair | 150.05 | 20.3 |

TABLE 8B
DMAMP-NH₃ in low DMAMP percentages

| | Denaturation | |
|---|---|---|
| Mole ratio | Temperature | |
| DMAMP/NH₃ | (° C.) | L* value |
| 100% DMAMP | 145.67 | 24.9 |
| 35/65 | 144.94 | 32.4 |
| 30/70 | 144.29 | 31.5 |
| 25/75 | 144.72 | 32.1 |
| 20/80 | 144.71 | 32.9 |
| 15/85 | 145.27 | 32.9 |
| 10/90 | 145.02 | 33.1 |
| 9/91 | 145.12 | 32.5 |
| 8/92 | 144.95 | 33.8 |
| 7/93 | 145.08 | 32.4 |
| 6/94 | 145.03 | 33.6 |
| 5/95 | 145.31 | 32.5 |
| 4/96 | 144.52 | 33.7 |
| 3/97 | 144.76 | 34.1 |
| 2/98 | 144.52 | 32.6 |
| 1/99 | 144.53 | 33.0 |
| 0.5/99.5 | 144.26 | 33.3 |
| 100% NH3 | 144.17 | 33.8 |
| Virgin hair | 148.12 | 20.3 |

TABLE 9
Tris-NH₃

| | Denaturation | |
|---|---|---|
| Mole ratio | Temperature | |
| Tris/NH₃ | (° C.) | L* value |
| 100% Tris | 148.61 | 28 |
| 90/10 | 148.29 | 30 |
| 67/33 | 148.00 | 32 |
| 50/50 | 147.61 | 33 |
| 33/67 | 146.92 | 33 |
| 10/90 | 147.63 | 33.5 |
| 100% NH₃ | 146.75 | 34 |
| Virgin Hair | 149.33 | 20.0 |

TABLE 10
Serinol-NH₃

| | Denaturation | |
|---|---|---|
| Mole ratio | Temperature | |
| Serinol/NH₃ | (° C.) | L* value |
| 100% Serinol | 143.50 | 31.2 |
| 90/10 | 143.70 | 32.3 |
| 67/33 | 143.53 | 32.4 |
| 50/50 | 143.34 | 32.2 |
| 33/67 | 143.54 | 34.2 |
| 10/90 | 143.61 | 34.4 |
| 100% NH₃ | 143.75 | 35.2 |
| Virgin hair | 146.14 | 19.9 |

TABLE 11
AMPD-Tris

| | Denaturation | |
|---|---|---|
| Mole ratio | Temperature | |
| AMPD/Tris | (° C.) | L* value |
| 100% AMPD | 146.78 | 31 |
| 90/10 | 146.86 | 31 |
| 67/33 | 146.75 | 29 |
| 50/50 | 147.33 | 29.2 |
| 33/67 | 148.17 | 29 |
| 10/90 | 148.59 | 28.5 |
| 100% Tris | 148.61 | 28 |
| Virgin hair | 150.75 | 20.3 |

TABLE 12
AEPD-Tris

| | Denaturation | |
|---|---|---|
| Mole ratio | Temperature | |
| AEPD/Tris | (° C.) | L* value |
| 100% AEPD | 142.76 | 30.4 |
| 90/10 | 142.28 | 30.3 |
| 67/33 | 142.90 | 31.6 |
| 50/50 | 143.45 | 31.1 |
| 33/67 | 144.54 | 29.8 |
| 10/90 | 145.89 | 29.8 |
| 100% Tris | 145.93 | 28.5 |
| Virgin hair | 146.70 | 20.4 |

TABLE 13
Serinol-Tris

| | Denaturation | |
|---|---|---|
| Mole ratio | Temperature | |
| Serinol/Tris | (° C.) | L* value |
| 100% Serinol | 144.78 | 31.2 |
| 90/10 | 144.89 | 31.2 |
| 67/33 | 145.53 | 31.4 |
| 50/50 | 145.72 | 30.6 |
| 33/67 | 145.81 | 28.1 |
| 10/90 | 146.91 | 28.4 |
| 100% Tris | 146.81 | 28.5 |
| Virgin hair | 146.70 | 20.4 |

The addition of AMPD, AEPD or Serinol to NH₃ lowered the denaturation temperature, but only slightly. The addition of DMAMP or Tris to NH₃ raised the denaturation temperature. Almost any amount of Tris lead to less damage than AMPD, AEPD or Serinol alone, and less damage than AMPD, AEPD or Serinol in combination with NH₃. In those combinations involving NH₃, the L* value decreased somewhat, indicating less efficient lifting than 100% NH₃. Tris tends to reduce the effectiveness of color lifting.

The denaturation temperature of each mixture of alkalizers varies approximately linearly with the relative concentration of each alkalizer. The combination of DMAMP-NH₃ is an exception. In that case, there are two clusters of data (see the FIGURE). A first cluster of temperature data exists between about 0 and 15 molar percentage of DMAMP. A second cluster exists between about 25 and 100 molar percentage of DMAMP. DMAMP-NH₃ exhibited a significant increase in denaturation temperature when going from 100% NH₃ to 0.5:99.5 (1:199 DMAMP:NH₃). This result was unexpected. This non-linear relationship is indicative of a range of molar percentages in which color lifting increases with little additional denaturation. We justifiably expect therefore, beneficial results for mole ratios in between 100% NH₃ and 3:7 (DMAMP:NH₃). For example, mole ratios (DMAMP:NH₃) of 1:199, 1:99, 1:90, 1:45, 1:30, 1:22.5, 1:18, 1:15, 1:12.9, 1:11.25, 1:3 and 1:2.5 are useful because of significantly less damage compared to 100% NH₃. A mole ratio between 1:3 and 1:99 is preferred; between 1:12.9 and 1:45 is more preferred; between 1:15 and 1:30 is still more preferred.

In total, the above data suggests the existence of preferred ranges of the relative concentration for each combination of alkalizers, depending on the effect sought. A summary of DSC results is shown in Table 14. Also shown are preferred ranges of the mole ratio for each combination of alkalizers, based on DSC results alone, and based on DSC and L* value results.

TABLE 14
Combinations of Alkalizers

| | | | Preferred |
|---|---|---|---|
| | | Preferred | range of |
| | | range of | mole ratio |
| | | mole ratio | based on |
| | Overall | based on | DSC and |
| Combination | DSC result | DSC alone | L* value |
| AMPD-NH₃ | certain | about 1:1 to | about 1:1 to |
| | concentrations | about 1:9 | about 1:9 |
| | showed | | |
| | improvement | | |
| | over 100% NH₃ | | |
| AEPD-NH₃ | up to 50% | about 1:1 to | about 1:1 to |
| | AEPD showed | about 1:3 | about 1:3 |
| | improvement | | |
| | over 100% | | |
| | NH₃ | | |
| DMAMP-NH₃ | all | about 1:199 to | about 1:199 to |
| | concentrations | about 100% | about 1:4 |
| | showed | DMAMP | |
| | improvement | | |
| | over 100% | | |
| | NH₃ | | |
| Tris-NH₃ | all | about 1:9 to | about 1:9 to |
| | concentrations | about 100% | about 2:1 |
| | showed | Tris | |
| | improvement | | |
| | over 100% | | |
| | NH₃ | | |
| Serinol-NH₃ | All | about 1:99 to | about 1:2.5 to |
| | concentrations | about 100% | about 1:9 |
| | were | Serinol | |
| | very similar | | |
| | to 100% | | |
| | NH₃ | | |
| AMPD-Tris | all | about 9:1 to | about 9:1 to |
| | concentrations | about 100% | about 1:99 |
| | of Tris showed | Tris | Tris |
| | improvement | | |
| | over 100% | | |
| | AMPD | | |
| AEPD-Tris | Almost all | about 9:1 to | about 9:1 to |
| | concentrations | about 100% | about 1:99 |
| | of Tris showed | Tris | Tris |
| | improvement | | |
| | over 100% | | |
| | AEPD | | |
| Serinol-Tris | all | about 9:1 to | about 9:1 to |
| | concentrations | about 100% | about 1:99 |
| | of Tris showed | Tris | Tris |
| | improvement | | |
| | over 100% | | |
| | Serinol | | |

### Cytotoxicity

The effects of alkalizer compositions on outer root sheath cells and on keratinocytes was evaluated. The MultiTox-Fluor Multiplex Cytotoxicity Assay (Promega Corp., Madison, WI) simultaneously measures two protease activities: one is a marker of cell viability, and the other is a marker of cytotoxicity. In the assays, the responses of outer root sheath cells and keratinocytes to various concentrations of alkalizers were measured, and IC₅₀, the concentration that provokes a mid-height response (midway between the baseline response and maximal response), was determined. In this study, a greater IC₅₀ value indicates that the alkalizer induces less stress on the cells. The results are shown in Table 15.

TABLE 15
Toxicity

| | IC₅₀ | |
|---|---|---|
| | Outer root | IC₅₀ |
| Alkalizer | sheath cells | Keratinocytes |
| NH₄•OH | 54.63 | 45.18 |
| (29% active) | | |
| (control) | | |
| MEA | 21.43 | 23.00 |
| AMP | 11.87 | 5.197 |
| DMAMP | 18.00 | 33.00 |
| AMPD | 103.6 | 106.1 |
| AEPD (97%) | 87.00 | 121.0 |
| 3-(dimethylamino)- | 124.0 | 212.0 |
| 1,2-propanediol | | |
| Isoserinol | 56.27 | 97.00 |

For either type of cell, conventional replacements for ammonia, MEA and AMP are more toxic to the cell than ammonium hydroxide. DMAMP is also more toxic than ammonium hydroxide, but less toxic than conventional replacement AMP, and less toxic to keratinocytes than MEA. Also, for either type of cell AMPD, AEPD, DMAPD and isoserinol are significantly less toxic than ammonium hydroxide.

### Tensile Strength

The effects of alkalizer compositions on the tensile strength of hair fibers was measured. Hair tresses were treated with the compositions shown in Tables 2 and 3, as described above. From each tress, multiple individual hair fibers were prepared for tensile analysis using brass crimps and a Diastron AAS 1600 (Diastron Ltd, UK) to thread and crimp the hair. The mean cross-sectional area of each fiber was determined using a laser micrometer FDAS 770 unit (Diastron Ltd, UK) at 24° C. and 55% relative humidity (RH). All hair fibers were stretched until break using a Diastron MTT 686 instrument with control unit UV 1000 (Diastron Ltd, UK). Final results were calculated by software analysis (UvW in 2.35.0000, Diastron, Ltd, UK). The average applied stress at break is shown in Table 16. A greater stress at break indicates that the hair fibers were less weakened by the applied alkalizer composition.

TABLE 16
Break Stress (gmf/sq micron)

| | NH3 | MEA | AMP | DMAMP | AMPD | AEPD | AP |
|---|---|---|---|---|---|---|---|
| Average | 2.19E−02 | 2.11E−02 | 2.10E−02 | 2.16E−02 | 2.19E−02 | 2.17E−02 | 2.11E−02 |
| Std. Dev. | 1.32E−03 | 1.47E−03 | 1.47E−03 | 1.67E−03 | 1.25E−03 | 1.42E−03 | 1.37E−03 |
| n | 49 | 49 | 49 | 49 | 46 | 48 | 49 |
| T-test | — | 0.004 | 0.001 | 0.234 | 0.990 | 0.332 | 0.004 |
| (vs. NH₃) | | | | | | | |

Break Stress (gmf/sq micron)

| | DMAP | DMAPD | Isoserinol | Serinol | DEA | TEA | Tris |
|---|---|---|---|---|---|---|---|
| Average | 2.21E−02 | 2.19E−02 | 2.17E−02 | 2.12E−02 | 2.13E−02 | 2.19E−02 | 2.18E−02 |
| StdDev | 1.59E−03 | 1.49E−03 | 9.83E−04 | 1.37E−03 | 1.66E−03 | 1.34E−03 | 1.66E−03 |
| n | 50 | 50 | 48 | 48 | 47 | 48 | 50 |
| T-test | 0.555 | 0.943 | 0.316 | 0.009 | 0.053 | 0.847 | 0.760 |
| (vs. NH₃) | | | | | | | |

The tensile data in Table 16 shows the average break stress for hair fiber after treatment with the alkalizing composition and oxidant. The samples are compared to NH₃ as the baseline. The break stress data indicate that, as a general trend that going from primary to tertiary amine decreases the amount of damage. For example, the tertiary amines (DMAMP, DMAP, DMAPD, TEA) cause a similar amount of damage or less damage than their primary amine analogs (AMP, AP, AMPD, MEA). Also, going from primary to secondary to tertiary decreases the amount of damage seen with MEA, DEA, and TEA. Furthermore, the data also show that the location and number of hydroxyl groups impacts the tensile strength, as seen when comparing A P vs AMP vs AMPD. The introduction of two hydroxyl groups (AMPD) surprisingly results in a less damaged hair fiber. A similar effect is observed with Tris which is a primary amine flanked by 3 hydroxyl groups. Both of these examples demonstrate fiber damage similar to NH₃ with comparable lift.

### Odor Evaluation

A professional perfumer ranked several of the alkalizer compositions represented in tables 1 and 2. The base formula (Table 1) with no alkalizer was used as a control. Among the primary alkanolamine compositions, the order from no malodor to strongest malodor was:

$$Control > AMPD = Serinol > Tris > MEA = AMP > AEPD > {NH}_{3}$$

Among the tertiary alkanolamine compositions, the order from no malodor to strongest malodor was:

$$Control > DMAMP > DMAPD > {NH}_{3}$$

All of the alkanolamine compositions that were evaluated have less malodor than the ammonium hydroxide composition. Among the primary amines, AMPD, Serinol and Tris performed better than MEA and AMP, two conventional ammonia replacements. The two tertiary amines tested did not perform as well as MEA and AMP.

### EXAMPLE

TABLE 17

| | Alkalizer | |
|---|---|---|
| | composition | Color lifting |
| | with dye | composition |
| INCI Name | Percent | Percent |
| Cetearyl alcohol | 2.00-10.00 | 2.00-10.00 |
| Steareth-21 | 1.00-4.00 | 1.00-4.00 |
| Sunflower oil | 2.00-3.00 | 2.00-3.00 |
| Decyl glucoside | 0.10-5.00 | 0.10-5.00 |
| Glycerin | 1.00-3.00 | 1.00-3.00 |
| Antioxidant | 0.50-1.00 | 0.50-1.00 |
| Tetrasodium EDTA | 0.30-0.50 | 0.30-0.50 |
| Oxidative dye | 0.01-25.0 | — |
| DMAMP compound | 0.50 | 0.50 |
| (80% active) | | |
| Ammonium hydroxide | 4.00 | 16.00 |
| (29% active) | | |
| Aroma | 0.01-1.00 | — |
| Water | q.s. | q.s. |

In summary, six of the eleven alkanolamines performed well in the DSC analysis, suggesting a reduction in hair fiber damage compared to ammonium hydroxide. Eight of the eleven alkanolamines performed comparably to NH₃ in the tensile test. In DSC analysis, ten of the eleven alkanolamines, when used as hair colorant alkalizers, demonstrated a reduction in hair fiber damage over MEA and AMP, conventional replacements for ammonium hydroxide. In tensile strength analysis, eight of the eleven alkanolamines, when used as hair colorant alkalizers, demonstrated a reduction in hair fiber damage over MEA and AMP. Three alkanolamines were better than ammonium hydroxide at lifting color from hair. One of these (isoserinol) was significantly better than AMP and about the same as MEA, at lifting hair color. Furthermore, combinations of the eleven alkalizers, with or without ammonium hydroxide, have proved beneficial in regard to reduction of hair damage, improved color lifting, or both. We have discovered that, for a mixture of two alkalizers, keratin denaturation temperature varies roughly linearly with the relative concentration of each alkalizer, except in the case of DMAMP-NH₃. This result was unexpected. This, combined with L* value measurements, suggests the existence of a preferred range of the relative concentration for each combination of alkalizers, and we have identified these.

Primary alkanolamines smell better than tertiary alkanolamines. For outer root sheath cells and keratinocyte, MEA, AMP and DMAMP are more toxic to the cell than ammonium hydroxide. However, for either type of cell AMPD, AEPD, DMAPD and isoserinol are significantly less toxic than ammonium hydroxide. Some or all of these properties may be beneficial in both oxidative and non-oxidative hair treatments.

In general, we have demonstrated the suitability of various alkanolamines for softening and swelling of the hair cuticle, for enabling penetration of reagents and hair-benefit actives into the cortex. These results are useful for various types of hair treatment applications, but we have specifically demonstrated the case of hair coloring treatments. We have shown that the use of ammonium hydroxide can be reduced or eliminated, depending on the type of hair coloring application.

### DISCUSSION

Based on observations, it may be surmised that intra-molecular hydrogen bonding within the alkanolamines results in conformations that provide some degree of stabilizing of the amine. Of foremost interest is the hydrogen bonding between the nitrogen atom and one or more hydroxyl groups of the alkanolamine. This type of intra-molecular bonding offers an explanation for the variation that we have observed in regards to hair fiber denaturation and color lifting. In general, the more hydrogen bonding in which the amine participates, the less damage experienced by the hair, but at the cost of less effective color lifting. However, of specific interest are those alkanolamine molecules wherein the electron bond donors are located at exactly two carbons away from the nitrogen atom. Those primary alkanolamines that have two or more hydroxyl groups located in C2 positions are useful alkalizers in hair treatment products. Also, secondary and tertiary alkanolamines that have at least one hydroxyl group located in a C2 position are useful.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An alkalizer composition that comprises:
one or more primary intermediates and/or couplers;
ammonia; and
2-dimethyl-amino-2-methyl-1-propanol,
wherein the mole ratio of 2-dimethyl-amino-2-methyl-1-propanol to ammonia is 1:3 to 1:99.

2. The alkalizer composition of claim 1 wherein the mole ratio of 2-dimethyl-amino-2-methyl-1-propanol to ammonia is 1:12.9 to 1:45.

3. The alkalizer composition of claim 1 wherein the mole ratio of 2-dimethyl-amino-2-methyl-1-propanol to ammonia is 1:15 to 1:30.

4. A hair-dye product comprising a first container and a second container, wherein:
the first container contains an alkalizer composition according to claim 1; and
the second container contains an oxidizing agent composition.

5. The hair-dye product of claim 4, wherein said product has a pH from 8 to 12.
